(19) United States
(12) Patent Application Publication
(10) Pub. No.: US 2022/0241145 A1
(43) Pub. Date: Aug. 4, 2022

(54) MEDICAL DEVICE FOR CONCURRENT ELECTRICAL STIMULATION

(71) Applicant: Cristina N. Medrano-Johnson, Mahomet, IL (US)

(72) Inventor: Cristina N. Medrano-Johnson, Mahomet, IL (US)

(21) Appl. No.: 17/583,575

(22) Filed: Jan. 25, 2022

Related U.S. Application Data
(60) Provisional application No. 63/144,623, filed on Feb. 2, 2021.

(51) Int. Cl. A61H 23/02; A61N 1/05; A61N 1/36

(52) U.S. Cl. CPC A61H 23/02; A61N 1/0551; A61N 1/36053; A61N 1/36135; A61H 2205/12; A61H 2201/5058; A61H 2201/5064; A61H 2203/0431; A61H 2201/5084; A61H 2201/10

(57) ABSTRACT

Concurrent electrical stimulation of a human being. Motion of a user is monitored based on motion and orientation signals received from motion sensors and a physical position of the user is determined based on the motion and orientation signals. A first electrode and/or one or more second electrodes are activated based on the determined physical position of the user. The first electrode is configured to provide a first electrical stimulation to a first nervous system of the user. The one or more second electrodes are configured to provide a second electrical stimulation to a second nervous system of the user. Vibrational electrodes are activated based on the determined physical position of the user. The vibration electrodes are configured to provide vibrational stimulation to an area of the user.

### RELATED APPLICATIONS

This application claims the benefit of U.S. Provisional Patent Application No. 63/144,623, filed on Feb. 2, 2021, the entire content of which is hereby incorporated by reference.

### FIELD

Embodiments described herein relate to device and methods for concurrent stimulation of a human nervous system.

### BACKGROUND

Various noninvasive methods of electrical stimulation have been studied for use in neurodegenerative disorders such as Parkinson's Disease (PD) and Multiple Sclerosis (MS). PD and MS exemplify disorders characterized not only with problems of motor and sensory components, but also an overall disconnect between the two. As a consequence, these disorders are present with both motor (somatic, autonomic, and enteric) and non-motor (general sensory, special sensory, and afferent autonomic) signs and symptoms. While somatic motor deficits are readily apparent, less visible non-motor complications are wide-ranging and debilitating, negatively impacting the quality of life. With PD for example, sleep disorders, mood changes, and constipation may be experienced in advance of the more familiar movement disorders of bradykinesia, resting tremors, rigidity, and postural instability. Autonomic motor dysfunction affects the heart and smooth muscles of the vasculature causing problems such as arrythmias and orthostatic intolerance. Pain, cognitive changes, and fatigue are disabling symptoms common to both PD and MS that are associated with sensory, motor, and autonomic dysfunction. Signs and symptoms are often addressed individually and often involve a delicate balance among treatments. The current approach to these neurodegenerative disorders includes pharmacologic, electrotherapeutic and rehabilitative modalities. However, treatments are not curative, and pharmacologic therapies may effectively oppose one another. Furthermore, sensorimotor disintegration associated with PD and MS is addressed in fragmented fashion which often neglects the underlying pathophysiologic process of inflammation. Invasive electrotherapeutic modalities (e.g., Deep Brain Stimulation for PD) are not appropriate for nonsurgical candidates and requires specific indications. While noninvasive electrotherapy is evolving, currently there is no comprehensive noninvasive stimulation tool or system of treatment that addresses multiple facets of neurodegenerative disorders like PD and MD.

Until recently, Galvanic Vestibular Stimulation (GVS) has been used primarily in research settings to elucidate the mechanisms underlying posture and gait. Conventional GVS applies an anode to one mastoid process and a cathode to the contralateral mastoid. In a healthy subject, when a direct current is applied, postural sway is induced in the direction of the anode. It delivers direct current (DC) transmastoidal electrical stimulation and has been shown to modulate balance, gait, and other motor dysfunctions in a variety of motor disorders, including PD and MS. Stochastic GVS has been shown in numerous studies to improve postural instability in patients with PD and MS. Recently, the role of the vestibular system has expanded to include functions outside of movement and balance (for example, cognition, affect, and autonomic control). Consequently, GVS has increasingly been used to study physiologic processes that are separate but related to movement.

Additionally, there has been interest in the use of vagal nerve stimulation (VNS) to treat a myriad of disorders. Surgically-implanted VNS systems were FDA-approved for treatment of epilepsy in the late 1990s and later treatment resistant depression in 2005. Concurrently, interest in noninvasive VNS methods manifested in the use of devices which stimulate externally accessible segments of the vagus nerve on the external ear (auricular branch of the vagus nerve) or the neck (cervical level vagus nerve). VNS has also been applied for the modulation of inflammatory and autoimmune disorders. Inflammation underlies the pathology in most neurodegenerative disorders while autoimmunity is thought to play a role in the perpetuation of the pathologic processes characteristic of PD and MS. VNS has been shown to improve non-motor symptoms commonly experienced in PD and MS which may be a result of its anti-inflammatory effect, through restoring autonomic balance by supporting the parasympathetic arm of the autonomic nervous system (ANS) or both. VNS also mediates neuroplasticity processes by increasing the receptive field of associated movements and sensory perceptions. Numerous studies have demonstrated that VNS concurrent with motor activity (e.g., stroke rehabilitation) facilitates learning, which manifests as faster return to function. Similarly, VNS paired with certain tones (special sensory stimulation) diminishes the perception of tinnitus by increasing the perception of sound in different frequencies. VNS effectively decreases the amount of cortical and subcortical neuronal structures that have been “assigned” to the pathologic sound. Thus, in disorders such as PD and MS which are characterized by sensory and motor deficits, sensorimotor disintegration and chronic inflammation, VNS presents a promising therapy with pleiotropic effects.

Adaptations to movement occur as a result of perceptions guided by sensory cues and prior knowledge. General sensory (somatosensory) and special sensory cues have long been known to affect motor function in PD and MS. Various sensory stimuli have been used to improve initiation and/or maintenance of movement these disorders. Numerous devices have been developed to deliver general or special sensory stimuli in specific and innovative ways to achieve benefits. However, the underlying mechanism is not fully understood and current devices functionally isolate stimulation targets.

### SUMMARY

Integrating signals from multiple sensory streams to plan, execute, and adjust movement appears effortless in a neurologically intact individual. The field of multisensory integration has established, in general, that the effect of bimodal stimulation is enhanced compared to unimodal stimulation as measured by response times. For example, response times decrease when both visual and auditory cues are delivered simultaneously compared to when delivered individually. While the underlying mechanism is yet to be fully understood, this may explain the improvement in speed and balance in some (visually intact) patients with PD when exposed to vibratory or auditory stimuli. While the enhancement effects of various physiologic processes (e.g., applying multimodal sensory cues, use of stochastic resonance to increase signal detection, etc.) can be used to improve motor function, they have not been incorporated into a comprehensive scheme to do so in PD and MS.

Despite promising benefits of various noninvasive stimulation methods that address sensor and motor disintegration, autonomic dysfunction, and chronic inflammation, there are no devices or systems which integrate these capabilities into one system. Use of such a device requires an understanding of electrode placement, appropriate stimulation parameters, and intended outcome. Furthermore, current prescriptive treatment methods are performed in clinic or laboratory setting with few systems designed for daily use. Currently, there are no devices which provide the option of either GVS and VNS in one system to overcome motor deficiencies resulting from sensorimotor integration dysfunction. As such, concurrent or interleaved GVS and VNS has also has not been performed using one system. Classically, GVS requires transcutaneous patch electrodes behind both ears on the mastoid processes while the ideal placement of the transcutaneous auricular VNS electrode is along the anterior aspect of the external ear in an area named the concha cavum. Others have also targeted the tragus during so-called transcutaneous auricular vagal nerve stimulation (tVNS). Moreover, VNS electrotherapy is typically administered unilaterally and GVS is applied bilaterally. GVS is traditionally applied as a constant DC in a way that can alter/improve one's postural sway in the lateral plane. Variations of the classical GVS montage have also been configured to induce anterior-posterior sway which has been used to improve camptocormia in PD. VNS electrodes are typically transdermal although percutaneous electrodes using fine needle arrays are also used. Percutaneous electrodes are rarely used for GVS.

One should note that while it is generally agreed that the vagal network or vestibular network are the targets of such stimulation, other structures are inevitably exposed to the applied energy. These may include underlying muscle spindle afferents, trigeminal nerve endings, microglial cells, lymphatic tissue, vascular and perivascular tissues, autonomic fibers, etc. Stimulation protocols for both VNS and GVS vary greatly due to the number of stimulation parameters (e.g., waveform, frequency, amplitude, duty cycle, treatment frequency and duration). VNS protocols may differ based on the intended effect. That is, the stimulation parameters for sensor or motor facilitation differ from the stimulation parameters to decrease inflammation. Likewise, stimulation protocols for GVS may differ based on the specific postural or movement deficiency present in the user. Stochastic GVS has also been shown to benefit patient's with PD using phenomenon of stochastic resonance or noise which facilitates sensory signals. VNS has been applied using various waveforms using alternating current (AC). Embodiments described herein allows for the electrical stimulation to modulate the activity of nervous and other tissues for the purpose of augmenting components of native sensorimotor pathways that are deficient in PD and MS. PD and MS exemplify disorders which have demonstrated the benefit of electrical stimulation to 1) presumptive nodes of integration of sensory stimuli which inform central nervous system motor output (i.e., brainstem structures) and 2) peripheral somatic and/or special sensory nerve end structures that transduce external stimuli into accurate representations to those centers. Specific targets of the former include, but are not limited to, cranial nerves and their nuclei (for example, vagal and vestibular), and other brainstem nuclei and regions which receive information from peripheral structures. Targets of the latter may include, but are not limited to, cutaneous sensory fibers afferents (for example, those translating pain, pressure, temperature stimuli) and muscle spindle afferents which relay proprioceptive information, and special sensory cues such as auditory or visual stimuli. Because GVS, VNS, and sensory stimulation have all demonstrated benefit to PD and MS, a device that integrates their capabilities is needed. Additionally, for greater benefit, such a device should be accessible for everyday use with clinical supervision available remotely.

Accordingly, one embodiment provides a device and methods to deliver electrical energy concurrently to the vestibular, vagal, and sensory pathways with stimulation paradigms that are determined by the status of the user as detected by relevant sensors or clinical indicators. The device and method may include a therapeutic system to treat conditions characterized by sensorimotor dysfunction that may also be present with dysautonomia and inflammation.

Another embodiment places one or more leads on the ears of the user to modulate activity of the vagal nervous system. One or more electrodes behind the ears modulate activity of the vestibular system. One or more electrodes throughout the body are applied to the skin (to stimulate somatosensory receptors) or about the surface of the head (to stimulate special sensory receptors). Multiple sensors throughout the body detect the user's movement, position, orientation, and autonomic status.

Another embodiment provides surface or minimally invasive percutaneous electrodes placed on an area of the external ear subserved by the auricular branch of the vagus nerve to modulate activity of the vagal nervous system. The device may provide alternating current in one or more stimulation programs to stimulate the vagal system. The device may be programmed to modulate the vagal system to facilitate a predetermined movement. The device may be programmed to modulate the vagal system to influence the autonomic nervous system status. The device may further be programmed to modulate inflammation as determined by clinical examination.

Another embodiment provides surface or minimally invasive percutaneous electrodes placed on an area behind the external ear to modulate activity of the vestibular system. The device may provide direct current stimulation in one or more stimulation programs to stimulate the vestibular system to 1) adjust posture in the lateral plane and 2) provide subsensory noise to facilitate signal detection. Additional electrodes may be placed on a central anterior or posterior aspect of the head and neck to adjust posture in the anteroposterior plane.

Another embodiment provides surface or minimally invasive percutaneous electrodes placed in an area to modulate activity of underlying neural tissue to induce somatosensory and/or special sensory perception. In one embodiment, surface electrodes on the surface of the bottom of the feet may deliver mechanical vibratory stimulation to the skin and underlying structures. The device may provide stimulation in one or more programs based on the desired movement. In one embodiment, the device provides stimulation to the bottom of both feet for a predetermined time prior or during detection of a predetermined movement wherein the stimulation settings may change in response to changes in said movement characteristics. In another embodiment, the device may provide special sensory stimulation. Surface electrodes on the head, lateral to the eyes may deliver stimulation to the skin and underlying optic nerve to create a visual stimulus. The device may be programmed to deliver such visual stimulation for a predetermined time prior to or during detection of a predetermined movement wherein the stimulation settings may change in response to changes in said movement characteristics.

Another embodiment includes a system of sensors placed on the surface of the skin to detect the physical status in terms of movement, position, and orientation of the user and parts of the user's body. Another embodiment includes a system of sensors placed on the surface of the skin to detect the physiologic and autonomic status of the user. The sensors may detect, for example, heart rate and rhythm, respiratory rate and volume, skin temperature, and/or transcutaneous oxygen pressure.

Another embodiment provides a method of concurrent electrical stimulation which utilizes data from physical and physiologic sensors to determine the stimulation parameters delivered by the electrodes targeting the vagal nervous system, vestibular nervous system, and various other sensory systems. The stimulation paradigms may be further delivered in an integrated and coordinated manner. One embodiment provides a method of monitoring and adjusting stimulation parameters remotely.

One embodiment provides a system for concurrent electrical stimulation of a user. The system includes a first electrode configured to provide a first electrical stimulation to a first nervous system of the user, one or more second electrodes configured to provide a second electrical stimulation to a second nervous system of the user, and one or more vibration electrodes configured to provide vibrational stimulation to an area of the user. The system includes one or more motion sensors configured to detect motion of the user and a controller electronically connected to the first electrode, the one or more second electrodes, the one or more vibration electrodes, and the one or more sensors. The controller is configured to monitor motion of the user based on motion and orientation signals received from the one or more motion sensors, determine, based on the motion signals, a physical position of the user, activate, based on the determined physical position of the user, at least one selected from a group consisting of the first electrode and the one or more second electrodes, and activate, based on the determined physical position of the user, the one or more vibration electrodes.

Another embodiment provides a method for concurrent electrical stimulation of a user. The method includes monitoring motion of the user based on motion and orientation signals received from one or more motion sensors, the one or more motion sensors configured to detect motion of the user, and determining, based on the motion signals, a physical position of the user. The method includes activating, based on the determined physical position of the user, at least one selected from a group consisting of a first electrode and one or more second electrodes, wherein the first electrode is configured to provide a first electrical stimulation to a first nervous system of the user, and wherein the one or more second electrodes are configured to provide a second electrical stimulation to a second nervous system of the user. The method includes activating, based on the determined physical position of the user, one or more vibration electrodes, wherein the one or more vibration electrodes are configured to provide vibrational stimulation to an area of the user.

Other aspects of various embodiments will become apparent by consideration of the detailed description and accompanying drawings.

### BRIEF DESCRIPTION OF THE DRAWINGS

FIG. 1 illustrates a treatment system according to some embodiments.

FIG. 2 illustrates a front view of a human ear showing features according to some embodiments.

FIG. 3 illustrates a side view of a human skull showing features according to some embodiments.

FIG. 4 illustrates a block diagram of a medical device of FIG. 1 according to some embodiments.

FIG. 5 illustrates a block diagram of a method performed by a controller of FIG. 4 according to some embodiments.

FIG. 6 illustrates a block diagram of a method performed by a controller of FIG. 4 according to some embodiments.

FIG. 7 illustrates a graph providing a range of intensities and frequencies of stimulation provided by a controller of FIG. 4 according to some embodiments.

FIG. 8 illustrates a block diagram of a method performed by a controller of FIG. 4 according to some embodiments.

### DETAILED DESCRIPTION

Before any embodiments are explained in detail, it is to be understood that the embodiments are not limited in their application to the details of the configuration and arrangement of components set forth in the following description or illustrated in the accompanying drawings. The embodiments are capable of being practiced or of being carried out in various ways. Also, it is to be understood that the phraseology and terminology used herein are for the purpose of description and should not be regarded as limiting. The use of “including,” “comprising,” or “having” and variations thereof are meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings.

In addition, it should be understood that embodiments may include hardware, software, and electronic components or modules that, for purposes of discussion, may be illustrated and described as if many of the components were implemented solely in hardware. However, one of ordinary skill in the art, and based on a reading of this detailed description, would recognize that, in at least one embodiment, the electronic-based aspects may be implemented in software (for example, stored on non-transitory computer-readable medium) executable by one or more electronic processors, for example, one or more microprocessors and/or application specific integrated circuits (“ASICs”). As a consequence, it should be noted that a plurality of hardware and software-based devices, as well as a plurality of different structural components, may be utilized to implement the embodiments. For example, “servers” and “computing devices” described in the specification can include one or more processing units, one or more computer-readable medium modules, one or more input/output interfaces, and various connections (for example, a system bus) connecting the components.

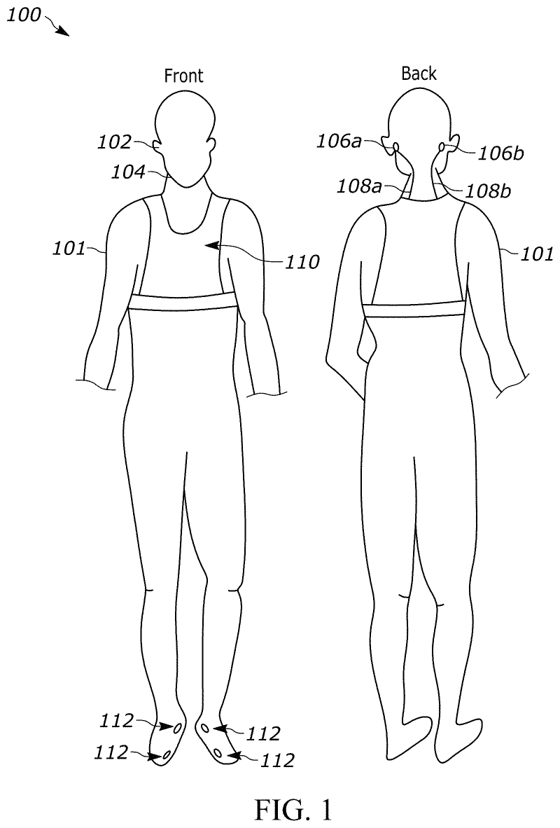

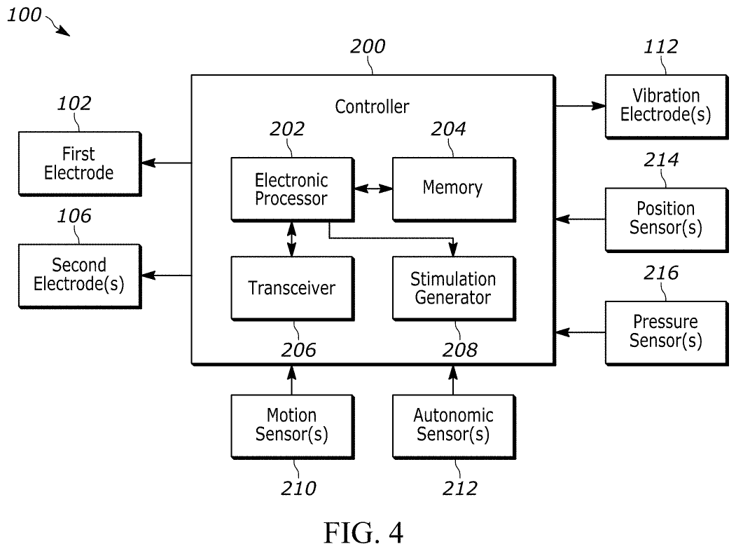

FIG. 1 provides a treatment system 100 according to some embodiments. The treatment system 100 includes a first electrode 102 and one or more second electrode(s) 106 (for example, a second electrode pair including a left electrode 106a and a right electrode 106b) coupled to a user 101. The first electrode 102 may be connected to a controller 200 (illustrated in FIG. 4) via a first link, for example, wire 104. The left electrode 106a and the right electrode 106b may be connected to the controller 200 via links, for example, a left wire 108a and a right wire 108b, respectively. In some embodiments, rather than using the first wire 104, the left wire 108a, and the right wire 108b, the first electrode 102, the left electrode 106a, and the right electrode 106b are wirelessly connected to the controller 200, for example, via Bluetooth™ connections. The controller 200 may be situated within a harness 110 worn by the user 101. The harness 110 may be implemented as a part of a jacket, a shirt, or other wearable clothing.

In some embodiments, the treatment system 100 includes one or more vibration electrode(s) 112 (for example, one or more piezoelectric actuators) connected to one or more feet of the user 101 (for example, the left foot and the right foot). The one or more vibration electrode(s) 112 may be configured to provide somatosensory stimulation (for example, vibrational stimulation) to the user 101. The one or more vibration electrode(s) 112 may be worn bilaterally such that both the left foot of the user 101 and the right food of the user 101 receive somatosensory stimulation. In some embodiments, the one or more vibration electrode(s) 112 are situated on another area of the user 101, such as an upper extremity and/or back of the user 101, hands of the user 101, or other section of the body in which vibration stimulation may be beneficial. In some embodiments, the one or more vibrational electrode(s) 112 are connected to the controller 200 via a wire (not shown). In other embodiments, the one or more vibrational electrode(s) 112 are wirelessly connected to the controller 200. The one or more vibrational electrode(s) 112 may further be wirelessly connected to the first electrode 102 and the one or more second electrode(s) 106.

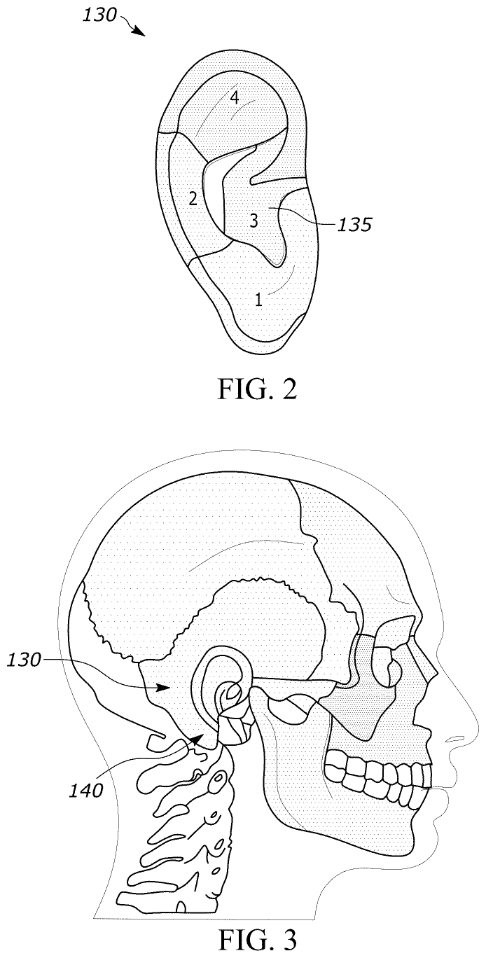

The first electrode 102 is configured to stimulate a first nervous system (for example, a vagal nervous system, a vagal nerve) of the user 101. For example, the first electrode 102 may be applied to the surface of an ear 130 to provide electrical stimulation to the area of skin subserved by the auricular branch of the vagus nerve 135 (FIG. 2). In some embodiments, a percutaneous needle electrode pierces the skin overlying the auricular branch of the vagus nerve 135 to stimulate the vagus nerve 135 and other underlying structures, as previously described. While illustrated in FIG. 1 as being within the right ear of the user 101, the first electrode 102 may be applied to either the right ear or the left ear of the user 101. Rather than coupling to the ear 130 as illustrated, in some embodiments, the first electrode 102 is a stimulating electrode implanted subdermally in the ear 130. Stimulating the auricular branch of the vagus nerve 135 using the first electrode 102 may assist in stabilizing autonomous functions of the user 101, such as blood pressure, heart rate, or the like. Another feature of the device allows a separate program of stimulating the auricular branch of the vagus nerve 135 with the first electrode 102, which may assist with signs and symptoms of inflammation. In some embodiments, the first electrode 102 provides alternating current (AC) stimulation.

The one or more second electrode(s) 106 are configured to stimulate a second nervous system (for example, a vestibular nervous system) of a user 101. For example, the one or more second electrode(s) 106 may be applied to the skin overlying the mastoid processes of the temporal bones 140 situated behind the ear 130 (FIG. 3). The one or more second electrode(s) 106 may be worn bilaterally, with one electrode behind the left ear and one electrode behind the right ear. Stimulating the vestibular nervous system using the one or more second electrode(s) 106 may assist in stabilizing balance and posture of the user 101. In some embodiments, the one or more second electrode(s) 106 provide direct current (DC) stimulation. In some embodiments, the one or more second electrode(s) 106 provide stochastic stimulation. In some embodiments, the first electrode 102 and the one or more second electrode(s) 106 are transcutaneous (surface) adhesive electrodes, percutaneous electrodes, or the like.

FIG. 4 illustrates a block diagram of the treatment system 100. In addition to the first electrode 102, the one or more second electrode(s) 106, and the one or more vibration electrode(s) 112, the treatment system 100 includes a controller 200, one or more motion sensor(s) 210, one or more autonomic sensor(s) 212, one or more position sensor(s) 214, and one or more pressure sensor(s) 216. The controller 200 includes, among other things, an electronic processor 202 (for example, a microprocessor, a microcontroller, or another suitable programmable device), a memory 204, a transceiver 206, and a stimulation generator 208. The controller 200 is electrically connected to the first electrode 102, the one or more second electrode(s) 106, the one or more vibration electrode(s) 112, the one or more motion sensor(s) 210, the one or more autonomic sensor(s) 212, the one or more position sensor(s) 214, and the one or more pressure sensor(s) 216.

The memory 204 is a non-transitory computer readable medium that includes, for example, a program storage area and a data storage area. The program storage area and the data storage area can include combinations of different types of memory, such as read-only memory (“ROM”), random access memory (“RAM”) (for example, dynamic RAM [“DRAM”], synchronous DRAM [“SDRAM”], etc.), electrically erasable programmable read-only memory (“EEPROM”), flash memory, a hard disk, an SD card, or other suitable magnetic, optical, physical, or electronic memory devices. The electronic processor 202 is connected to the memory 204 and executes software instructions that are capable of being stored in a RAM of the memory 204 (for example, during execution), a ROM of the memory 204 (for example, on a generally permanent basis), or another non-transitory computer readable medium such as another memory or a disc. Software included in the implementation of the treatment system 100 can be stored in the memory 204 of the controller 200. The software includes, for example, firmware, one or more applications, program data, filters, rules, one or more program modules, and other executable instructions. The controller 200 is configured to retrieve from the memory 204 and execute, among other things, instructions related to the control processes and methods described herein. In other constructions, the controller 200 includes additional, fewer, or different components.

The transceiver 206 may be configured to communicate (for example, transmit and receive communication signals, data, information, and the like) with a device external to the treatment system 100. For example, the transceiver 206 transmits and receives information from a mobile device (for example, a cellular device, a personal laptop, a tablet, or the like) of the user 101. In some embodiments, the transceiver 206 communicates with a mobile device of a physician associated with the user 101. The transceiver 206 may provide operational information associated with the user 101 to a server, such as a hospital server, a medical insurance server, or the like.

The one or more motion sensor(s) 210 are configured to provide motion signals associated with motion of the user 101 (for example, detect motion of the user 101). The one or more motion sensor(s) 210 may include, for example, an accelerometer, a gyroscope, or the like. The one or more motion sensor(s) 210 may be situated at, for example, a chest of the user 101, the legs of the user 101, the arms of the user 101, the knees of the user 101, or the like. The controller 200 may use the motion signals to determine a position of the user 101, such as whether the user 101 is sitting or standing. In some embodiments, the controller 200 is coupled to one or more position sensor(s) 214 such that the controller 200 determines a position of the user 101 regardless of movement. Additionally, the controller 200 may use the motion signals from the one or more motion sensor(s) 210 and/or the position signals from the one or more position sensor(s) 214 to determine movement of the user 101, such as whether the user 101 is stationary, walking, or running. In some embodiments, the controller 200 uses the motion signals to monitor the balance and/or the posture of the user 101.

In some embodiments, the controller 200 receives pressure signals from one or more pressure sensor(s) 216 connected to the controller 200. The one or more pressure sensor(s) 216 may be placed on the plantar (bottom) aspect of the feet of the user 101. The controller 200 may further determine a position of the user 101 based on the pressure signals. For example, the pressure signals include a high voltage when the user 101 is standing compared to when the user 101 is sitting or laying down. Additionally, one or more pressure sensor(s) 216 may be situated on other parts of the body, such as the hands of the user 101. When on the hands, the one or more pressure sensor(s) 216 may allow the controller 200 to monitor characteristics of the user 101 such as grip strength.

The one or more autonomic sensor(s) 212 are configured to provide autonomic signals associated with autonomic functions of the user 101 (for example, detect autonomic functions of the user 101). The one or more autonomic sensor(s) 212 may include, for example, a heart rate sensor, a respiratory monitor, a blood pressure sensor, or the like. The controller 200 may control the first electrode 102, the one or more second electrode(s) 106, and/or the one or more vibration electrode(s) 112 based on the autonomic signals, as described in more detail below.

The stimulation generator 208 generates alternating current (AC) and/or direct current (DC) stimulation for the first electrode 102 and the one or more second electrode(s) 106. In some embodiments, the stimulation generator 208 generates a pulse width modulated (PWM) signal provided to the first electrode 102 and the one or more second electrode(s) 106. The electronic processor 202 may control the stimulation generator 208 based on the motion signals from the one or more motion sensor(s) 210 and the autonomic signals received from the one or more autonomic sensor(s) 212.

The controller 200 controls stimulation of the user 101 through the first electrode 102, the one or more second electrode(s) 106, and the vibration electrode(s) 112 based on various statuses and functions of the user 101. For example, the one or more motion sensor(s) 210, the one or more autonomic sensor(s) 212, the one or more position sensor(s) 214, and the one or more pressure sensor(s) 216 provide the controller 200 with information on the user 101, varying from movement and orientation to autonomic indices and somatosensory signals, as provided by the various sensors.

By stimulating the vagal nervous system with the first electrode 102 and the vestibular nervous system with the one or more second electrode(s) 106, the treatment system 100 can assist the user 101 with stability as they move about their day. As the user 101 moves to and from sitting and standing positions, or increase and decrease the speed at which they move, electrical stimulation to the corresponding nervous system may help with balance and improve mobility. Further stimulation via the one or more vibration electrode(s) 112 may further assist with these motions. Additionally, the stimulation parameters may depend on detected autonomic functions of the user 101 and customized by individual physicians. For example, a minimum and maximum amount of stimulation provided by the first electrode 102, the one or more second electrode(s) 106, and the one or more vibration electrode(s) 112 may all be customized by the treating physician.

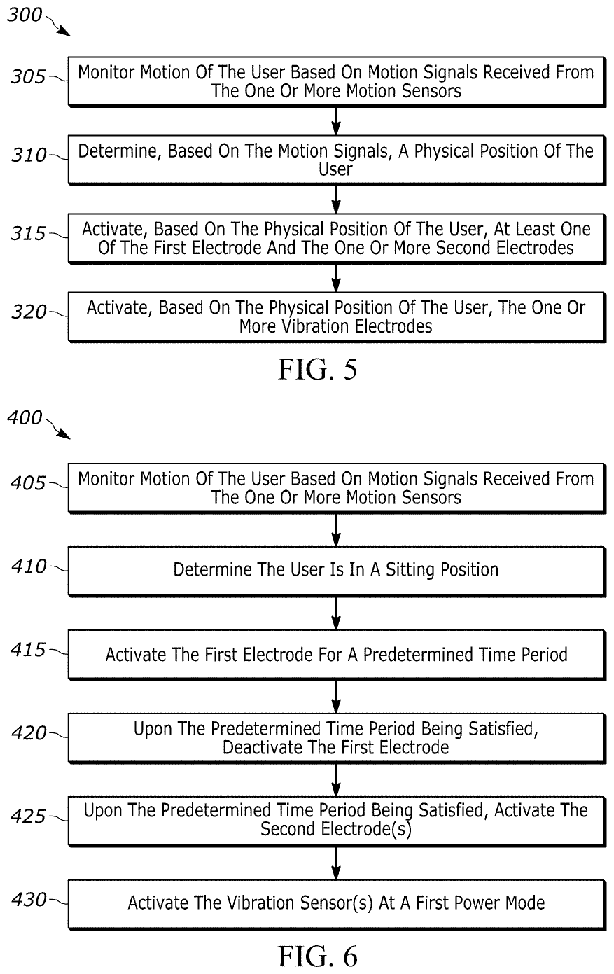

For example, FIG. 5 provides a method 300 for controlling the first electrode 102, the one or more second electrode(s) 106, and the one or more vibration electrode(s) 112 based on the motion signals received from the one or more motion sensor(s) 210. At block 305, the controller 200 monitors motion of the user 101 based on motion signals received from the one or more motion sensor(s) 210. The motion signals may be, for example, indicative of a position of the user 101, such as a sitting position, a supine position, or a standing position. In some embodiments, the motion signals from the one or more motion sensor(s) 210 provide the controller 200 with motion information over a period of time. The motion signals may indicate the user 101 transitioning from a sitting position to a standing position, a standing position to a sitting position, or the like. Additionally, the motion signals may indicate the user 101 walking or running, rather than remaining in a position. In this manner, the controller 200 constantly monitors movement of the user 101. In some embodiments, the user 101 provides the controller 200 with an input indicative of upcoming movement. For example, the user 101 provides an input on a mobile device coupled to the controller 200 or a touch display of the controller 200. The input may indicate an upcoming transition from a supine position to a sitting position, from a sitting position to a standing position, or the like.

At block 310, the controller 200 determines, based on the motion signals received from the one or more motion sensor(s) 210, a physical position of the user 101. The physical position of the user 101 may be, for example, a supine position, a sitting position, a standing position, a walking motion, or a running motion. In some embodiments, the controller 200 determines the physical position of the user 101 based on position signals received from the one or more position sensor(s) 214. At block 315, the controller 200 activates, based on the physical position of the user 101, at least one of the first electrode 102 and the one or more second electrode(s) 106, as described in more detail below. At block 320, the controller 200 activates, based on the physical position of the user 101, the one or more vibration electrode(s) 112, as described in more detail below.

The controller 200 controls the first electrode 102, the one or more second electrode(s) 106, and the vibration electrode(s) 112 based on movement, orientation, autonomic indices, and somatosensory (e.g., pressure) signals associated with the user 101. FIG. 6, for example, provides a method 400 for when the controller 200 determines the user 101 is in the sitting position. At block 405, the controller 200 monitors motion of the user 101 based on motion signals received from the one or more motion sensor(s) 210 or position signals received from the one or more position sensor(s) 214, as previously described. At block 410, the controller 200 determines the user 101 is in, or is entering, a sitting position. In some embodiments, the controller 200 determines the user 101 is in, or entering, a supine position.

At block 415, the controller 200 activates the first electrode 102 for a predetermined time period. Activating the first electrode 102 to stimulate the vagal nervous system may assist the user 101 with balance and stability as they transition to the sitting or supine position. Additionally, stimulating the vagal nervous system may assist the user 101 with regulating changes in heart rate or blood pressure as they transition to the sitting or supine position. In some embodiments, inflammation of the user 101 is monitored. If an inflammation indicator or other monitored characteristic indicative of the inflammation exceeds a threshold, the first electrode 102 is activated to assist with or relieve related symptoms. At block 420, upon the predetermined time period being satisfied, the controller 200 deactivates the first electrode 102. Accordingly, the vagal nervous system may be stimulated for only the predetermined time period. In some embodiments, the stimulation provided by the first electrode 102 decreases over the predetermined period. For example, the stimulation may decrease linearly over the duration of the predetermined time period from a first level to a second level. The stimulation provided by the first electrode 102 may begin to decrease once the predetermined time period has been satisfied. Additionally, rather than a linear decrease, in some embodiments, the stimulation provided by the first electrode 102 may decrease according to another function, such as an exponential decay.

At block 425, upon the predetermined time period being satisfied, the controller 200 activates the one or more second electrode(s) 106. Activating the one or more second electrode(s) 106 to stimulate the vestibular nervous system may assist the user 101 with posture and balance as they move from a sitting position to a standing position, from a standing position to a sitting position, and the like. For example, in some embodiments, the controller 200 controls the left electrode 106a and the right electrode 106b as an anode/cathode pair. For example, if the user 101 is swaying to the left as they are switching positions, the controller 200 provides stimulation to the right electrode 106b, which acts as an anode. The left electrode 106a then acts as a cathode. By providing the stimulation with the right electrode 106b, the user 101 sways to the right, and therefore moves to a more center, or upright, position. Alternatively, if the user 101 is swaying to the right as they are switching positions, the controller 200 provides stimulation to the left electrode 106a, which now acts as an anode. The right electrode 106b then acts as a cathode, such that the user 101 sways to the left, and therefore moves to the upright position.

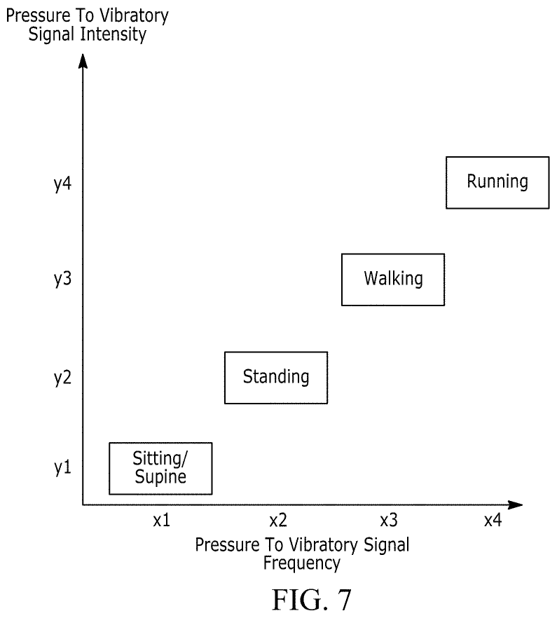

At block 430, the controller 200 activates the one or more vibration electrode(s) 112 at a first mode. For example, FIG. 7 provides a graph illustrating a relationship between the intensity of the vibration provided by the one or more vibration electrode(s) 112 and the frequency of the vibration provided by the one or more vibration electrode(s) 112 with respect to the position of the user 101. For example, when in the sitting position or the supine position, the vibration provided by the one or more vibration electrode(s) 112 has an intensity of y1 and a frequency of x1. In some embodiments, the intensity of the vibration provided by the one or more vibration electrode(s) 112 decreases over time. In some embodiments, the controller 200 activates the one or more vibration electrode(s) 112 in response to the user input indicating an upcoming transition. The one or more vibration electrode(s) 112 may then remain on until a predetermined time after the transition occurs. Alternatively, the one or more vibration electrode(s) 112 may decrease or stop stimulation based on the one or more motion sensor(s) 210 and the one or more pressure sensor(s) 216 indicating an adequate walking velocity and cadence. While FIG. 7 provides an embodiment that is a generally linear relationship between the intensity and frequency of vibration, the actual relationship may differ in other embodiments and/or from user to user.

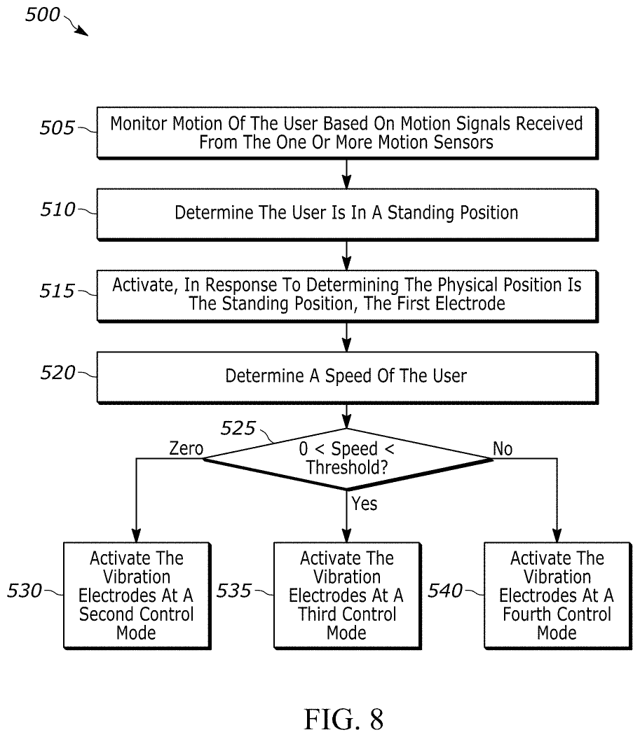

FIG. 8 provides a method 500 for when the controller 200 determines the user 101 is in a standing position. At block 505, the controller 200 monitors motion of the user 101 based on motion signals received from the one or more motion sensor(s) 210 or position signals received from the one or more position sensor(s) 214, as previously described. At block 510, the controller 200 determines the user 101 is in a standing position. In some embodiments, the controller 200 determines the user 101 is entering the standing position, such as moving from the sitting position to the standing position.

At block 515, the controller 200 activates, in response to determining the physical position is the standing position, the first electrode 102. The parameters of the stimulation provided by the first electrode 102 may be dependent on the autonomic signals provided by the one or more autonomic sensor(s) 212. Additionally, the stimulation parameters provided by the first electrode 102 when the user 101 is standing may differ from the stimulation parameters provided by the first electrode 102 when the user 101 is in the sitting position or supine position. In some embodiments, in response to determining the physical position is the standing position, the controller 200 activates the one or more vibration electrode(s) 112.

At block 520, the controller 200 determines a speed of the user 101 based on the motion signals received from the one or more motion sensor(s) 210. For example, the controller 200 may determine the user 101 is moving at a speed between 0 miles per hour and 6 miles per hour. The speed may be determined by the speed at which the legs of the user 101 are moving. At block 525, the controller 200 compares the speed to a speed threshold.

If the speed is equal to zero, the controller 200 proceeds to block 530, determines the user 101 remains in the standing position, and activates the one or more vibration electrode(s) 112 at a second control mode. In some embodiments, the second control mode has a greater vibration intensity and a greater vibration frequency than the first control mode. For example, as illustrated in FIG. 7, when in the standing position, the vibration provided by the one or more vibration electrode(s) 112 has an intensity of y2 and a frequency of x2. If the speed is greater than zero but less than the speed threshold, the controller 200 proceeds to block 535, determines the user 101 is walking (for example, in a walking position), and activates the one or more vibration electrode(s) 112 at a third control mode. In some embodiments, the third control mode has a greater vibration intensity and a greater vibration frequency than the second control mode. For example, as illustrated in FIG. 7, when in the walking position, the vibration provided by the one or more vibration electrode(s) 112 has an intensity of y3 and a frequency of x3. If the speed is greater than the threshold, the controller 200 proceeds to block 540, determines the user 101 is running (for example, in a running position), and activates the one or more vibration electrode(s) 112 at a fourth control mode. In some embodiments, the fourth control mode has a greater vibration intensity and a greater vibration frequency than the third control mode. For example, as illustrated in FIG. 7, when in the running position, the vibration provided by the one or more vibration electrode(s) 112 has an intensity of y4 and a frequency of y4. In some embodiments, the speed threshold is approximately 4 miles per hour.

In some embodiments, the controller 200 determines whether the user 101 is sitting, standing, walking, or running based on autonomic signals received from the one or more autonomic sensor(s) 212. For example, the heart rate of the user 101 may be compared to a heart rate threshold rather than, or in conjunction with, a speed threshold. Should the heart rate of the user 101 be approximately 60-70 beats per minute (BPM), the controller 200 may determine the user 101 is sitting. Should the heart rate of the user 101 be approximately 70-80 BPM, the controller 200 may determine the user 101 is standing. Should the heart rate of the user 101 be approximately 80-120 BPM, the controller 200 may determine the user 101 is walking. Should the heart rate of the user 101 be greater than 120 BPM, the controller 200 may determine the user 101 is running.

Intensity and frequency (for example, a power level) of the stimulation provided by the first electrode 102 and the one or more second electrode(s) 106 may be adjusted by the controller 200 based on the motion signals from the one or more motion sensor(s) 210 and the autonomic signals from the one or more autonomic sensor(s) 212. For example, the power level of stimulation provided by the one or more second electrode(s) 106 may increase should the user 101 show significant signs of swaying from an upright position. The treatment time of stimulation provided by the first electrode 102 may increase should the heart rate of the user 101 vary from an expected heart rate based on the position of the user 101.

In some embodiments, the controller 200 manages a log of activity of the user 101. For example, the controller 200 may store information on when each of the first electrode 102, the left electrode 106a, and the right electrode 106b were activated and deactivated. The log may include measurements of the one or more autonomic sensor(s) 212 alongside the activity of the first electrode 102 and the one or more second electrode(s) 106, allowing a viewer of the log to view how the autonomic system of the user 101 responded to stimulation. The log may be transmitted to a mobile device, such as a personal cellular device, personal computer, or the like, such that a viewer, such as a physician, can view the log. The physician may then alter operation of the first electrode 102, the one or more second electrode(s) 106, and the one or more vibration electrode(s) 112 based on the log. Accordingly, the treatment system 100 can be modified based on the individual user 101 and how they react to stimulation. Modifying operation may occur remotely, from the physician's lab or office, or via Bluetooth during an appointment.

Additionally, the methods described herein are not limited to what is described. For example, the order in which operation is presented may be altered, and steps may occur simultaneously. While embodiments primarily describe the controller 200 controlling the first electrode 102, the one or more second electrode(s) 106, and the one or more vibration electrode(s) 112 based on motion and position of the user 101, treatment may also be based on autonomic functions and treatment as prescribed by a physician.

Thus, embodiments provide, among other things, systems and methods for concurrent electrical stimulation of a human nervous system. Various features and embodiments are set forth in the following claims.

What is claimed is:

1. A system for concurrent electrical stimulation of a user, the system comprising:
a first electrode configured to provide a first electrical stimulation to a first nervous system of the user;
one or more second electrodes configured to provide a second electrical stimulation to a second nervous system of the user;
one or more vibration electrodes configured to provide vibrational stimulation to an area of the user;
one or more motion sensors configured to detect motion of the user; and
a controller electronically connected to the first electrode, the one or more second electrodes, the one or more vibration electrodes, and the one or more motion sensors, the controller configured to:
monitor motion of the user based on motion and orientation signals received from the one or more motion sensors;
determine, based on motion signals received from the motion sensors, a physical position of the user;
activate, based on the determined physical position of the user, at least one selected from a group consisting of the first electrode and the one or more second electrodes; and
activate, based on the determined physical position of the user, the one or more vibration electrodes.

2. The system of claim 1, wherein the controller is further configured to:
determine, based on the motion signals, the physical position as a sitting position;
activate, in response to determining the physical position as the sitting position;
the first electrode for a predetermined period of time; and
activate, in response to determining the physical position as the sitting position, the one or more second electrodes.

3. The system of claim 2, wherein the controller is further configured to:
deactivate, upon the predetermined period of time being satisfied, the first electrode.

4. The system of claim 2, wherein the controller is further configured to:
activate, in response to determining the physical position as the sitting position, the one or more vibration electrodes at a first control mode.

5. The system of claim 1, wherein the first nervous system is a vagal nervous system, and wherein the second nervous system is a vestibular nervous system.

6. The system of claim 1, wherein the controller is further configured to:
determine, based on the motion signals, the physical position is a standing position;
determine, based on the motion signals, a speed of the user;
activate, in response determining the physical position is the standing position, the first electrode;
compare the speed of the user to a speed threshold;
activate, in response to the speed of the user being less than the speed threshold, the one or more vibration electrodes at a first control mode; and
activate, in response to the speed of the user being greater than or equal to the speed threshold, the one or more vibration electrodes at a second control mode.

7. The system of claim 1, further comprising one or more autonomic sensors configured to detect one or more autonomic functions of the user.

8. The system of claim 7, wherein the controller is further configured to:
adjust, based on the one or more autonomic functions of the user, one or more stimulation parameters of the first electrode; and
adjust, based on the one or more autonomic functions of the user, one or more stimulation parameters of the one or more second electrodes.

9. The system of claim 1, wherein the one or more motions sensors includes at least one selected from a group consisting of a gyroscope and an accelerometer.

10. The system of claim 1, wherein the controller is further configured to:
activate, based on the determined physical position of the user, the one or more vibration electrodes at a first vibration level; and
decrease, over a first period of time, the first vibration level to a second vibration level.

11. The system of claim 1, wherein the controller is further configured to activate, based on a measured indicator of inflammation, the first electrode.

12. A method for concurrent electrical stimulation of a user, the method comprising:
monitoring motion of the user based on motion and orientation signals received from one or more motion sensors, the one or more motion sensors configured to detect motion of the user
determining, based on the motion signals, a physical position of the user;
activating, based on the determined physical position of the user, at least one selected from a group consisting of a first electrode and one or more second electrodes, wherein the first electrode is configured to provide a first electrical stimulation to a first nervous system of the user, and wherein the one or more second electrodes are configured to provide a second electrical stimulation to a second nervous system of the user, and
activating, based on the determined physical position of the user, one or more vibration electrodes, wherein the one or more vibration electrodes are configured to provide vibrational stimulation to an area of the user.

13. The method of claim 12, further comprising:
determining, based on the motion signals, the physical position as a sitting position,
activating, in response to determining the physical position as the sitting position, the first electrode for a predetermined period of time, and
activating, in response to determining the physical position as the sitting position, the one or more second electrodes.

14. The method of claim 12, wherein the first nervous system is a vagal nervous system, and wherein the second nervous system is a vestibular nervous system.

15. The method of claim 12, further comprising:
determining, based on the motion signals, the physical position is a standing position,
determining, based on the motion signals, a speed of the user,
activating, in response to determining the physical position is a standing position, the first electrode,
comparing the speed of the user to a speed threshold,
activating, in response to the speed of the user being less than the speed threshold, the one or more vibration electrodes at a first control mode, and
activating, in response to the speed of the user being greater than or equal to the speed threshold, the one or more vibration electrodes at a second control mode.

16. The method of claim 12, further comprising:
receiving one or more autonomic signals from one or more autonomic sensors, the autonomic signals associated with one or more autonomic functions of the user.

17. The method of claim 16, further comprising:
adjusting, based on the one or more autonomic functions of the user, one or more stimulation parameters of the first electrode, and
adjusting, based on the one or more autonomic functions of the user, one or more stimulation parameters of the one or more second electrodes.

18. The method of claim 12, wherein the one or more motion sensors include at least one selected from a group consisting of a gyroscope and an accelerometer.

19. The method of claim 12, further comprising:
activating, based on the determined physical position of the user, the one or more vibration electrodes at a first vibration level, and
decreasing, over a first period of time, the first vibration level to a second vibration level.

20. The method of claim 12, further comprising activating, based on a measured indicator of inflammation, the first electrode.
